(19) United States
(12) Patent Application Publication
(10) Pub. No.: US 2022/0305092 A1
(43) Pub. Date: Sep. 29, 2022

(54) MEDICAMENT AND FOOD FOR PREVENTING OR TREATING NOVEL CORONAVIRUS PNEUMONIA COVID-19 AND USE THEREOF

(71) Applicants: GUANGZHOU CENTURY CLINICAL RESEARCH CO., LTD, Guangzhou (CN); GUANGZHOU XIN-CHUANGYI BIOPHARMACEUTICAL CO., LTD, Guangzhou (CN); GUANGZHOU WELMAN NEW DRUG R&D CO., LTD., Guangzhou (CN); XIANGBEI WELMAN PHARMACEUTICAL CO., LTD, Changsha, Hunan (CN); NANJING KANGFUSHUN PHARMACEUTICAL CO., LTD, Nanjing, Jiangsu (CN)

(72) Inventors: Mingjie SUN, Guangzhou (CN); Tianyu SUN, Guangzhou (CN); Changqing LI, Guangzhou (CN)

(73) Assignees: GUANGZHOU CENTURY CLINICAL RESEARCH CO., LTD, Guangzhou (CN); GUANGZHOU XIN-CHUANGYI BIOPHARMACEUTICAL CO., LTD, Guangzhou (CN); GUANGZHOU WELMAN NEW DRUG R&D CO., LTD., Guangzhou (CN); XIANGBEI WELMAN PHARMACEUTICAL CO., LTD, Changsha, Hunan (CN); NANJING KANGFUSHUN PHARMACEUTICAL CO., LTD, Nanjing, Jiangsu (CN)

(21) Appl. No.: 17/441,885

(22) PCT Filed: May 9, 2020
(86) PCT No.: PCT/CN2020/089422

§ 371 Date: Sep. 22, 2021

(30) Foreign Application Priority Data
Apr. 27, 2020 (CN) 202010341811.3

(51) Int. Cl. A61K 38/47; A61P 31/14; A61K 31/704; A61K 31/215; A61K 31/513; A61K 31/427; A61K 31/4965; A61K 31/7056; A61K 31/675; A61K 31/4706; A61K 31/4045; A61K 45/06; A61K 38/21

(52) U.S. Cl. CPC A61K 38/47; A61P 31/14; A61K 31/704; A61K 31/215; A61K 31/513; A61K 31/427; A61K 31/4965; A61K 31/7056; A61K 31/675; A61K 31/4706; A61K 31/4045; A61K 45/06; A61K 38/21

(57) ABSTRACT

The use of lysozyme or combinations comprising the lysozyme in preparing a medicament for preventing or treating the novel coronavirus pneumonia COVID-19 and in preparing a food for assisting in preventing or treating the novel coronavirus pneumonia COVID-19 is disclosed. The lysozyme or the combination of the lysozyme, as provided by the present invention, can effectively inhibit cell damage caused by the novel coronavirus, inhibit the inflammatory factors expression induced by the novel coronavirus, ameliorate airway obstruction and decreased expiratory volume caused by lung lesions, and ameliorate intestinal barrier dysfunction. The lysozyme or the combination of the lysozyme has good effects on patients with high viral load, respiratory distress, impaired intestinal function, etc., and can reduce the novel coronavirus infection rate and the incidence rate of severe novel coronavirus pneumonia.

### TECHNICAL FIELD

The present invention relates to the field of medicine, and in particular, to a medicament and food for preventing or treating the novel coronavirus pneumonia COVID-19 and the use thereof.

### BACKGROUND ART

Some medical institutions in Wuhan, China, received patients with a pneumonia of unknown cause in December, 2019, which was identified as a pneumonia caused by a novel coronavirus COVID-19 (novel coronavirus pneumonia). Subsequently, an epidemic began to spread, and the novel coronavirus pneumonia outbreak occurred in Wuhan, China, and became a global pandemic. The novel coronavirus is highly contagious, and the mortality rate of patients with severe novel coronavirus pneumonia is high. At present, there have been cumulatively no less than 2.8 million patients with confirmed infections globally, and the cumulative death toll is no less than 200,000. The disease has caused an unprecedented huge loss of lives and properties to the human society.

COVID-19 is a new virus belonging to the family of coronaviridae. COVID-19 has certain similarities to SARS coronavirus, but also has obvious differences, especially in terms of viral gene sequence, asymptomatic carriers, infectivity, clinical symptoms, histopathology etc. Pathological observations have shown that the degree of pulmonary fibrosis in patients with the novel coronavirus pneumonia is less than that in patients with the atypical pneumonia SARS; however, pulmonary hyperplasia and obstruction in the patients with the novel coronavirus pneumonia are severer than those in the patients with the atypical pneumonia SARS.

Currently, some drugs which may be used for treating the novel coronavirus pneumonia have been recommended all over the world. However, many of them are intended for emergency use or compassionate use, and there are still many shortcomings. For example, broad-spectrum antiviral drugs have no obvious activity against COVID-19, and many drugs for the symptomatic treatment of the viral pneumonia also have poor effects. There are also a large number of drugs which may inhibit the virus, but have obvious toxicity. Therefore, there is an urgent need for a safe and effective medicament for treating the novel coronavirus pneumonia.

### SUMMARY OF THE INVENTION

A first objective of the present invention is to provide a use in preparing a medicament for preventing or treating a novel coronavirus pneumonia. In order to achieve the objective, the technical solution used in the present invention is:

the use of lysozyme or a combination comprising the lysozyme in preparing a medicament for preventing or treating the novel coronavirus pneumonia COVID-19.

In some embodiments, the combination further comprises glycyrrhizic acid or a salt thereof.

In some embodiments, the combination further comprises an additional ingredient useful for preventing or treating the novel coronavirus pneumonia COVID-19.

In some embodiments, the additional ingredient useful for preventing or treating the novel coronavirus pneumonia COVID-19 is one or more selected from an interferon, oseltamivir or a salt thereof, lopinavir, ritonavir, favipiravir, ribavirin, remdesivir, chloroquine or a salt thereof, hydroxychloroquine or a salt thereof, arbidol or a salt thereof, an interleukin inhibitor, a tumor necrosis factor inhibitor, a janus kinase inhibitor, a glucocorticoid, a protease inhibitor, and an intestinal microecological regulator.

In some embodiments, the protease inhibitor may be selected from ulinastatin, sivelestat, nafamostat, or tranexamic acid.

In some embodiments, the interleukin inhibitor may be selected from tocilizumab.

In some embodiments, the tumor necrosis factor inhibitor may be selected from adalimumab, infliximab, or etanercept.

In some embodiments, the janus kinase inhibitor may be selected from tofacitinib or baricitinib.

In some embodiments, the intestinal microecological regulator may be selected from prebiotics or probiotics.

In some embodiments, the lysozyme and the glycyrrhizic acid or the salt thereof in the combination may be combined in various manners. In some embodiments, the lysozyme and the glycyrrhizic acid or the salt thereof may be combined in such a manner that they exist separately in different pharmaceutical products (such as combined packaging or combined medication). In other embodiments, the lysozyme and the glycyrrhizic acid or the salt thereof may also be combined in such a manner that they coexist in the same pharmaceutical product (a compound preparation).

In some embodiments, the lysozyme and the additional ingredient useful for preventing or treating the novel coronavirus pneumonia COVID-19 in the combination may be combined in various manners. The lysozyme and the additional ingredient may be combined in such a manner that they exist separately in different pharmaceutical products (combined packaging or combined medication), or may also be combined in such a manner that they coexist in the same pharmaceutical product (a compound preparation).

In some embodiments, the dosage form for the medicament may be selected from various dosage forms suitable for medicinal use, and may be, for example, an oral dosage form, an injection dosage form, an inhalation dosage form, etc.

In some embodiments, the medicament may be presented as preparations with various release forms, such as a normal release preparation, a delayed release preparation, a sustained release preparation, or a controlled release preparation.

In some embodiments, the medicament further comprises a pharmaceutically applicable excipient.

In some embodiments, the weight ratio of the lysozyme to the glycyrrhizic acid or the salt thereof in the combination may be 100:1 to 1:100, preferably 100:1 to 2:1, more preferably 100:1 to 5:1, and further preferably 100:1 to 10:1. In some preferred solutions, the lysozyme and the glycyrrhizic acid or the salt thereof may have a significant synergistic effect.

In some embodiments, the daily dosage of the lysozyme may be 0.5 g to 20 g.

In some embodiments, the lysozyme may be administered during the non-sleeping time at a dosage frequency of once every 1 to 24 hours, such as once every 1 hour, every 2 hours, every 4 hours, every 6 hours, every 8 hours, every 12 hours, or every 24 hours.

A second objective of the present invention is to provide a use in preparing a food for assisting in preventing or treating a novel coronavirus pneumonia. In order to achieve the objective, the technical solution used in the present invention is:

the use of lysozyme or a combination comprising the lysozyme in preparing a food for assisting in preventing or treating the novel coronavirus pneumonia COVID-19.

In some embodiments, the food is a dietary supplement or a food for special medical use.

In some embodiments, the combination further comprises glycyrrhizic acid or a salt thereof.

In some embodiments, the food further comprises an additive suitable for food.

In some embodiments, the additive may be a preservative, a colorant, a flavoring agent, a spice, an antistaling agent, an antioxidant, an emulsifier, a thickening agent, a carbohydrate, a fat, a vitamin, an amino acid, a trace element, a protein, etc.

In some embodiments, the lysozyme and the glycyrrhizic acid or the salt thereof in the combination may be combined in various manners. The lysozyme and the glycyrrhizic acid or the salt thereof may be combined in such a manner that they exist separately in different food products (such as combined packaging, combined use), or may also be combined in such a manner that they coexist in the same food product (e.g. a compound food).

In some embodiments, the weight ratio of the lysozyme to the glycyrrhizic acid or the salt thereof in the combination may be 100:1 to 1:100, preferably 100:1 to 2:1, more preferably 100:1 to 5:1, and further preferably 100:1 to 10:1. In some preferred solutions, the lysozyme and the glycyrrhizic acid or the salt thereof may have a significant synergistic effect.

In some embodiments, the daily dosage of the lysozyme may be 0.5 g to 20 g.

In some embodiments, the lysozyme may be administered during the non-sleeping time at a dosage frequency of once every 1 to 24 hours, such as once every 1 hour, every 2 hours, every 4 hours, every 6 hours, every 8 hours, every 12 hours, or every 24 hours.

A third objective of the present invention is to provide a medicament for preventing or treating novel coronavirus pneumonia. In order to achieve the objective, the technical solution used in the present invention is:

a medicament for preventing or treating the novel coronavirus pneumonia COVID-19, wherein the medicament comprises lysozyme.

In some embodiments, the medicament further comprises glycyrrhizic acid or a salt thereof.

In some embodiments, the medicament further comprises an additional ingredient useful for preventing or treating the novel coronavirus pneumonia COVID-19.

In some embodiments, the additional ingredient useful for preventing or treating the novel coronavirus pneumonia COVID-19 is one or more selected from an interferon, oseltamivir or a salt thereof, lopinavir, ritonavir, favipiravir, ribavirin, remdesivir, chloroquine or a salt thereof, hydroxychloroquine or a salt thereof, arbidol or a salt thereof, an interleukin inhibitor, a tumor necrosis factor inhibitor, a janus kinase inhibitor, a glucocorticoid, a protease inhibitor, and an intestinal microecological regulator.

In some embodiments, the dosage form of the medicament may be selected from various dosage forms suitable for medicinal use, such as an oral dosage form, an injection dosage form, and an inhalation dosage form.

In some embodiments, the medicament may be presented as preparations with various release forms, such as a normal release preparation, a delayed release preparation, a sustained release preparation, or a controlled release preparation.

In some embodiments, the medicament further comprises a pharmaceutically applicable excipient.

In some embodiments, the weight ratio of the lysozyme to the glycyrrhizic acid or the salt thereof in the medicament may be 100:1 to 1:100, preferably 100:1 to 2:1, more preferably 100:1 to 5:1, and further preferably 100:1 to 10:1. In some preferred solutions, the lysozyme and the glycyrrhizic acid or the salt thereof may have a significant synergistic effect.

A fourth objective of the present invention is to provide a food for assisting in preventing or treating a novel coronavirus pneumonia. In order to achieve the objective, the technical solution used in the present invention is:

a food for assisting in preventing or treating the novel coronavirus pneumonia COVID-19, wherein the food comprises lysozyme.

In some embodiments, the food further comprises glycyrrhizic acid or a salt thereof.

In some embodiments, the food further comprises an additive suitable for food.

In some embodiments, the additive may be a preservative, a colorant, a flavoring agent, a spice, an antistaling agent, an antioxidant, an emulsifier, a thickening agent, a carbohydrate, a fat, a vitamin, an amino acid, a trace element, a protein, etc.

In some embodiments, the weight ratio of the lysozyme to the glycyrrhizic acid or the salt thereof in the food may be 100:1 to 1:100, preferably 100:1 to 2:1, more preferably 100:1 to 5:1, and further preferably 100:1 to 10:1. In some preferred solutions, the lysozyme and the glycyrrhizic acid or the salt thereof may have a significant synergistic effect.

A fifth objective of the present invention is to provide a method for preventing or treating novel coronavirus pneumonia. In order to achieve the objective, the technical solution used in the present invention is:

a method for preventing or treating the novel coronavirus pneumonia COVID-19, the method comprising administering a medicament to a subject in need thereof, wherein the medicament comprises an effective amount of lysozyme, or the medicament comprises an effective amount of a combination comprising the lysozyme.

In some embodiments, the subject is tested positive for COVID-19 novel coronavirus nucleic acid.

In some embodiments, the subject is clinically diagnosed with the novel coronavirus pneumonia COVID-19.

In some embodiments, in addition to being tested positive for the COVID-19 novel coronavirus nucleic acid, or being clinically diagnosed with the novel coronavirus pneumonia COVID-19, the subject further suffers from increased airway resistance or decreased expiratory volume.

In some embodiments, in addition to being tested positive for the COVID-19 novel coronavirus nucleic acid, or being clinically diagnosed with the novel coronavirus pneumonia COVID-19, the subject further suffers from impaired intestinal barrier function.

In some embodiments, the combination further comprises glycyrrhizic acid or a salt thereof.

In some embodiments, the combination further comprises an additional ingredient useful for preventing or treating the novel coronavirus pneumonia COVID-19.

In some embodiments, the additional ingredient useful for preventing or treating the novel coronavirus pneumonia COVID-19 is one or more selected from an interferon, oseltamivir or a salt thereof, lopinavir, ritonavir, favipiravir, ribavirin, remdesivir, chloroquine or a salt thereof, hydroxychloroquine or a salt thereof, arbidol or a salt thereof, an interleukin inhibitor, a tumor necrosis factor inhibitor, a janus kinase inhibitor, a glucocorticoid, a protease inhibitor, and an intestinal microecological regulator.

In some embodiments, the lysozyme and the glycyrrhizic acid or the salt thereof in the combination may be combined in various manners. In some embodiments, the lysozyme and the glycyrrhizic acid or the salt thereof may exist separately in different pharmaceutical products, for example, applied in a combined packaging or combined medication manner. In other embodiments, the lysozyme and the glycyrrhizic acid or the salt thereof may coexist in the same pharmaceutical product, for example, administered as a compound medicament.

In some embodiments, the lysozyme and the additional ingredient useful for preventing or treating the novel coronavirus pneumonia COVID-19 in the combination may be combined in various manners. In some embodiments, the lysozyme and the additional ingredient may exist separately in different pharmaceutical products, for example, applied in a combined packaging or combined medication manner. In other embodiments, the lysozyme and the additional ingredient may coexist in the same pharmaceutical product, for example, administered as a compound medicament.

In some embodiments, for the administration of the medicament, there may be a variety of methods, such as oral administration, injection, and inhalation.

In some embodiments, the medicament may be presented as preparations with various release forms, such as a normal release preparation, a delayed release preparation, a sustained release preparation, and a controlled release preparation.

In some embodiments, the medicament may further comprise a pharmaceutically applicable excipient.

In some embodiments, the weight ratio of the lysozyme to the glycyrrhizic acid or the salt thereof in the combination may be 100:1 to 1:100, preferably 100:1 to 2:1, more preferably 100:1 to 5:1, and further preferably 100:1 to 10:1. In some preferred solutions, the lysozyme and the glycyrrhizic acid or the salt thereof may have a significant synergistic effect.

In some embodiments, the daily dosage of the lysozyme may be 0.5 g to 20 g.

In some embodiments, the medicament may be administered during the non-sleeping time at a dosage frequency of once every 1 to 24 hours, such as once every 1 hour, every 2 hours, every 4 hours, every 6 hours, every 8 hours, every 12 hours, or every 24 hours.

A sixth objective of the present invention is to provide a method for assisting in preventing or treating novel coronavirus pneumonia. In order to achieve the objective, the technical solution used in the present invention is:

a method for assisting in preventing or treating the novel coronavirus pneumonia COVID-19, the method comprising administering a food to a subject in need thereof, wherein the food comprises an effective amount of lysozyme, or the food comprises an effective amount of a combination comprising the lysozyme.

In some embodiments, the food is a dietary supplement or a food for special medical use.

In some embodiments, the subject is tested positive for COVID-19 novel coronavirus nucleic acid.

In some embodiments, the subject is clinically diagnosed with the novel coronavirus pneumonia COVID-19.

In some embodiments, in addition to being tested positive for COVID-19 novel coronavirus nucleic acid, or being clinically diagnosed with the novel coronavirus pneumonia COVID-19, the subject further suffers from increased airway resistance or decreased expiratory volume.

In some embodiments, in addition to being tested positive for the COVID-19 novel coronavirus nucleic acid, or being clinically diagnosed with the novel coronavirus pneumonia COVID-19, the subject further suffers from impaired intestinal barrier function.

In some embodiments, the combination further comprises glycyrrhizic acid or a salt thereof.

In some embodiments, the lysozyme and the glycyrrhizic acid or the salt thereof in the combination may be combined in various manners. In some embodiments, the lysozyme and the glycyrrhizic acid or the salt thereof exist separately in different food products, for example, combined in a combined packaging or combined medication manner. In other embodiments, the lysozyme and the glycyrrhizic acid or the salt thereof coexist in the same food product, for example, combined as a compound food.

In some embodiments, the weight ratio of the lysozyme to the glycyrrhizic acid or the salt thereof in the combination may be 100:1 to 1:100, preferably 100:1 to 2:1, more preferably 100:1 to 5:1, and further preferably 100:1 to 10:1. In some preferred solutions, the lysozyme and the glycyrrhizic acid or the salt thereof may have a significant synergistic effect.

In some embodiments, the food may be administrated by various manners, such as oral administration, nasogastric feeding, injection, inhalation etc.

In some embodiments, various release forms may be applied for the food, such as normal release, delayed release, sustained release, and controlled release.

In some embodiments, the food further comprises an additive suitable for food, such as preservative, a colorant, a flavoring agent, a spice, an antistaling agent, an antioxidant, an emulsifier, a thickening agent, a carbohydrate, a fat, a vitamin, an amino acid, a trace element, or a protein.

In some embodiments, the daily dosage of the lysozyme may be 0.5 g to 20 g.

In some embodiments, the food may be administered during the non-sleeping time at a dosage frequency of once every 1 to 24 hours, such as once every 1 hour, every 2 hours, every 4 hours, every 6 hours, every 8 hours, every 12 hours, or every 24 hours.

### Beneficial Effects of the Invention

The lysozyme or the combination of the lysozyme as provided by the present invention can effectively inhibit the cell damage caused by the COVID-19 novel coronavirus, inhibit the inflammatory response caused by the COVID-19 novel coronavirus, ameliorate airway obstruction and decreased expiratory volume caused by lung lesions, and improve the intestinal barrier function. It has a good therapeutical effect on high viral load, respiratory distress, impaired intestinal function, etc. in patients with the novel coronavirus pneumonia COVID-19. It can reduce both the novel coronavirus infection rate and the incidence rate of severe novel coronavirus pneumonia. Moreover, in view of the extremely high safety of lysozyme, the lysozyme and the combination of the lysozyme also present great value for prevention against the novel coronavirus pneumonia in undiagnosed people at high risk of new coronavirus infection.

### DETAILED DESCRIPTION OF EMBODIMENTS

The present invention is described in detail hereinafter with reference to particular embodiments. It is to be understood that the content of the particular embodiments is illustrative, rather than restrictive, that is, they do not limit the content of the present invention in any way.

### Definition

The Chinese term “rongjunmei” refers to lysozyme. The lysozyme of the present invention may be either a lysozyme derived from an animal, a plant, or a microorganism, or a recombinant product of a natural lysozyme. For example, the lysozyme may be a hen egg white lysozyme, a human lysozyme, a recombinant human lysozyme, a bacteriophage lysozyme, etc. The lysozyme of the present invention includes a pharmaceutical salt thereof, such as a hydrochloride, chloride, sulfate and amino acid salt thereof. Lysozyme was first discovered by Fleming as an endogenous enzyme widely present in organisms. Lysozyme has been approved for use in food or medicine worldwide. In the United States lysozyme has been Generally Recognized As Safe (GRAS). Lysozyme has been permitted by WHO, many European countries, Japan, and China for use as food additives, and has been approved for use in medicine in China, Japan, Singapore, and other countries. At present, lysozyme is known to have a relatively strong antiviral ability against herpes viruses.

“COVID-19 novel coronavirus”, namely 2019 Novel Coronavirus, is a virus discovered in 2019, and is a new virus belonging to the family of coronaviridae.

“Novel coronavirus pneumonia COVID-19” is a pneumonia caused by an infection with the COVID-19 novel coronavirus. The pathological characteristics, clinical manifestations, and diagnostic criteria of the novel coronavirus pneumonia COVID-19 are described in detail in the “Guidelines for the Diagnosis and Treatment of Novel Coronavirus (2019-nCoV) Infection” issued by the National Health Commission of the PRC. Many characteristics of the novel coronavirus pneumonia COVID-19 are obviously different from those of bacterial pneumonia, viral pneumonia, and atypical pneumonia (severe acute respiratory syndrome, SARS).

The present invention is further illustrated hereinafter with reference to embodiments; however, the embodiments do not limit the solutions and effects of the present invention.

### Example 1: Use of Lysozyme in Patients with Novel Coronavirus Pneumonia and People at High Risk of Novel Coronavirus Infection

During the novel coronavirus pneumonia epidemic in China in 2019, our company donated twice to Hubei Province medicines worth more than 2 million RMB, including antimicrobial medicines such as lysozyme lozenges and enteric-coated lysozyme tablets. We found that these medicines played a positive role in fighting against the novel coronavirus pneumonia epidemic.

Nine patients who were newly diagnosed with the novel coronavirus pneumonia were administered with the lysozyme lozenges and/or enteric-coated lysozyme tablets that we provided, at a dose ranging from 0.5 g to 2 g per day. The patients insisted on the administration almost every day, and as a result, only one patient progressed to a severe condition, while the other eight patients were all mildly ill and subsequently recovered. The rate of progression to severe condition was calculated to be 11.1%.

Three people who lived and traveled in high-risk areas during the epidemic insisted on taking lysozyme lozenges and/or enteric-coated lysozyme tablets every day, at a dose ranging from 1 g to 1.5 g per day, and they were not found to be infected with the novel coronavirus after the nucleic acid test when they returned to work, with the infection rate being 0%.

As reported in “The Epidemiological Characteristics of an Outbreak of 2019 Novel Coronavirus Diseases (COVID-19) in China”, the proportion of patients with severe and critical conditions, among patients with the novel coronavirus pneumonia, was up to 18.5%, and after progression to severe and critical conditions, the mortality rate could be even closer to 50%. By comparison, we found that the administration of a lysozyme preparation could greatly reduce the severe disease rate and thereby reduce the mortality rate, and thus, it had a very positive effect on preventing and treating the novel coronavirus pneumonia epidemic. In addition, the lysozyme preparation also prevented people at high risk of novel coronavirus infection from being infected, which was of great value for controlling the spread and recurrence of the epidemic.

### Example 2: In Vitro Test of Lysozyme Against Novel Coronavirus

The effect of the lysozyme against COVID-19 novel coronavirus was studied using African green monkey kidney cells.

Drugs: Lysozyme and monoammonium glycyrrhizinate.

Cells: African green monkey kidney cells (VeroE6 cells).

Virus: COVID-19, with a titer of 100 TCID₅₀.

Culture: 100 μL of VeroE6 cells with a concentration of 2×10⁵ cells/mL were added to each well of a sterile 96-well culture plate, and cultured at 37° C. for 24 hours. The culture plate was divided, by wells, into a blank control group, a virus control group, a low-dose lysozyme group, a medium-dose lysozyme group, a high-dose lysozyme group, a low-dose monoammonium glycyrrhizinate group, and a high-dose monoammonium glycyrrhizinate group, with 3 wells in each group. Except the blank control group, 100 μL/well of a 100 TCID₅₀ virus solution was added to each group, and after adsorption in a 5% CO₂ incubator at 37° C. for 2 hours, the cell culture solution in the culture plate was discarded.

Addition of drugs: 100 μL/well of drug solutions were added to the wells of the groups according to the following dosages: the low-dose lysozyme group (500 μg/ml), the medium-dose lysozyme group (1000 μg/ml), the high-dose lysozyme group (2000 μg/ml), the low-dose monoammonium glycyrrhizinate group (1000 μg/ml), and the high-dose monoammonium glycyrrhizinate group (2000 μg/ml), and incubation was then performed at 37° C. for 4 days.

Observation of cytopathic effects: After incubation was completed, the cytopathic effect (CPE) was observed under an optical microscope. The degree of the cytopathic effect found in the cells was recorded according to the following six levels: “−” which means no cytopathic effect, “±” which means that the cytopathic effect is less than 10%, “+” which means that the cytopathic effect is about 25%, “++” which means that the cytopathic effect is about 50%, “+++” which means that the cytopathic effect is about 75%, and “++++” which means that the cytopathic effect is no less than 75%. The inhibition rate of the drug in each group on the cytopathic effect caused by the virus was calculated. The higher the inhibition rate, the better the effect.

Results: The main results of the test were listed in Table 1. The test results showed that the lysozyme significantly inhibited the cytopathic effect caused by the COVID-19 virus. The glycyrrhetate had a relatively weak inhibition effect on the COVID-19 virus.

TABLE 1
Inhibition rate of lysozyme on cytopathic
effect caused by COVID-19 virus

| | Inhibition |
|---|---|
| Drug (concentration) | rate (%) |
| Lysozyme (500 μg/ml) | 15.00 ± 8.66 |
| Lysozyme (1000 μg/ml) | 46.67 ± 15.28 |
| Lysozyme (2000 μg/ml) | 78.33 ± 2.89 |
| Monoammonium glycyrrhizinate (1000 μg/ml) | 23.35 ± 5.43 |
| Monoammonium glycyrrhizinate (2000 μg/ml) | 29.42 ± 7.16 |

### Example 3: In Vitro Test of Combinations Comprising the Lysozyme Against Novel Coronavirus and Inflammation Caused by Novel Coronavirus

The inhibition effect of combinations comprising the lysozyme on cytopathic effect and inflammation caused by the COVID-19 virus was studied.

The drugs, cells, viruses etc. were all the same as those in Example 2.

The grouping in a culture plate and the dosages of the drugs were as follows: a blank control group, a virus control group, a lysozyme group (350 μg/ml), lysozyme and monoammonium glycyrrhizinate group I (100 μg/ml+1 μg/ml), lysozyme and monoammonium glycyrrhizinate group II (100 μg/ml+5 μg/ml), lysozyme and monoammonium glycyrrhizinate group III (100 μg/ml+10 μg/ml), and lysozyme and monoammonium glycyrrhizinate group IV (100 μg/ml+50 μg/ml).

The method for observing the cytopathic effect was the same as that in Example 2, and the test results were shown in Table 2. In addition, cells without any cytopathic effect were collected, RNA was extracted, and the relative expression levels of inflammatory factors such as TNF-α and IL-6 were determined by means of a quantitative PCR method. The test results were shown in Table 3.

TABLE 2
Inhibition effects of combinations comprising lysozyme
on cytopathic effect caused by COVID-19 virus

| | Inhibition |
|---|---|
| Drug (concentration) | rate (%) |
| Lysozyme (350 μg/ml) | 6.67 ± 1.56 |
| Lysozyme + monoammonium glycyrrhizinate | 38.20 ± 8.11## |
| (100 μg/ml + 1 μg/ml) | |
| Lysozyme + monoammonium glycyrrhizinate | 49.32 ± 9.45## |
| (100 μg/ml + 5 μg/ml) | |
| Lysozyme + monoammonium glycyrrhizinate | 23.68 ± 4.31## |
| (100 μg/ml +10 μg/ml) | |
| Lysozyme + monoammonium glycyrrhizinate | 8.20 ± 2.27 |
| (100 μg/ml + 50 μg/ml) | |

Note:
comparing the drug combination groups with the lysozyme group, p < 0.05 (#), and p < 0.01 (##).

TABLE 3
Effects of combinations comprising lysozyme on cell inflammatory
factors expression induced by COVID-19 virus

| | Relative | Relative |
|---|---|---|
| | expression | expression |
| | quantity of | quantity of |
| Group | TNF-α mRNA | IL-6 mRNA |
| Blank control group | 1.01 ± 0.06 | 1.08 ± 0.07 |
| Virus control group | 3.61 ± 0.29 | 7.68 ± 0.50 |
| Lysozyme group (350 \|μg/ml) | 3.12 ± 0.26* | 6.78 ± 0.30** |
| Lysozyme + monoammonium | 2.25 ± 0.36**## | 4.30 ± 0.21**## |
| glycyrrhizinate group I | | |
| (100 μg/ml + 1 μg/ml) | | |
| Lysozyme + monoammonium | 2.13 ± 0.14**## | 3.47 ± 0.25**## |
| glycyrrhizinate group II | | |
| (100 μg/ml + 5 μg/ml) | | |
| Lysozyme + monoammonium | 2.65 ± 0.26**# | 5.74 ± 0.31**## |
| glycyrrhizinate group III | | |
| (100 μg/ml +10 μg/ml) | | |
| Lysozyme + monoammonium | 3.15 ± 0.10 | 6.45 ± 0.43** |
| glycyrrhizinate group IV | | |
| (100 μg/ml + 50 μg/ml) | | |

Note:
comparing the drug groups with the virus control group, p < 0.05 (*) and p < 0.01 (**). Comparing the drug combination groups with the lysozyme group, p < 0.05 (#) and p < 0.01 (##).

It could be seen from Tables 2 and 3 that the lysozyme and the drug combinations comprising the lysozyme had a better inhibition effect on the cytopathic effect caused by the COVID-19 virus, and could significantly reduce the increase of the cell inflammatory factors, as caused by the COVID-19 virus. Compared with the lysozyme alone, effects of drug combinations were obviously better, which manifested in a significant increase in the inhibition rate, a significant reduction in the levels of inflammatory factors, and a substantial reduction in the dosage of the drugs. It was suggested that there was a strong synergistic effect between glycyrrhetate and lysozyme, and in particular, when the dosage of the glycyrrhetate was relatively low, the synergistic effect was better.

### Example 4: Effects of Lysozyme and the Lysozyme Combinations on Airway Resistance and Lung Function of Guinea Pigs which had Inhaled Smoke

The smoke-induced model was a classic animal model for chronic obstructive pulmonary disease. After inhaling smoke for a long period of time, animals could suffer from increased mucus secretion, airway obstruction, lung function decline, etc. The effect of the lysozyme on airway obstruction was evaluated by means of the model establishment method in the present experiment.

1. Test Drugs

Lysozyme, monoammonium glycyrrhizinate, and monopotassium glycyrrhizinate.

2. Animals and Grouping

Male Hartley guinea pigs, weighing 400 to 500 g, were randomly divided into a blank group, a model group, a lysozyme oral administration group (200 mg/kg), a lysozyme inhalation group (2 mg/kg), lysozyme+monoammonium glycyrrhizinate inhalation group (2 mg/kg+0.1 mg/kg), lysozyme+monopotassium glycyrrhizinate oral administration group I (200 mg/kg+20 mg/kg), and lysozyme+monopotassium glycyrrhizinate oral administration group II (200 mg/kg+40 mg/kg), with eight animals in each group.

3. Model Establishment

The animals in each group except the blank group were exposed to cigarette smoke by using a smoke exposure system for oro-nasal inhalation only, such that the animals inhaled smoke of five cigarettes within 40 minutes per day, for 5 exposure days per week, for 12 consecutive weeks.

4. Preparation and Administration of Drugs

The lysozyme and monoammonium glycyrrhizinate used for inhalation administration were respectively crushed into powders with a particle size of less than 10 μm. The lysozyme and monopotassium glycyrrhizinate used for oral administration were respectively dissolved in normal saline.

The animals in each group except the blank group and the model group were administered once a day for 4 consecutive weeks from the 8th week after the model establishment began. The inhalation administration groups were administered by means of an oro-nasal inhalation exposure system, while the oral administration groups were administered intragastrically. Animals in the blank group and the model group were administered intragastrically with normal saline per day.

5. Test

Airway resistance measurement and 100 ms (millisecond) expiratory volume measurement were performed after administration.

The animals were placed in a double chamber plethysmography system, and after the animals were left in a tranquil state for 10 minutes, the specific airway resistance (sRaw) was measured. The animals were anesthetized by intramuscular injection of ketamine, then a catheter was then inserted into the trachea, and the forced expiratory volume in 100 ms (FEV 100) of the animals was measured through a lung function testing system.

6. Test Results and Evaluation

The results of the airway resistance measurement and the 100 ms expiratory volume measurement of the animals were as shown in FIG. 4.

TABLE 4
Effects of lysozyme and lysozyme combinations on airway
resistance and 100 ms expiratory volume of animals

| | Airway | 100 ms |
|---|---|---|
| | resistance | expiratory |
| Group | (cmH2O · s) | volume (mL) |
| Blank group | 1.43 ± 0.24 | 11.86 ± 1.24 |
| Model group | 4.72 ± 0.38 | 4.18 ± 1.26 |
| Lysozyme oral | 3.79 ± 0.30** | 5.79 ± 2.03 |
| administration group | | |
| Lysozyme inhalation group | 3.10 ± 0.14** | 7.68 ± 1.36** |
| Lysozyme + | 2.52 ± 0.22**&& | 9.04 ± 0.84** |
| monoammonium | | |
| glycyrrhizinate | | |
| inhalation group | | |
| Lysozyme + | 2.78 ± 0.17**## | 8.05 ± 1.56**# |
| monopotassium | | |
| glycyrrhizinate oral | | |
| administration group I | | |
| Lysozyme + | 3.47 ± 0.28** | 5.89 ± 1.01 |
| monopotassium | | |
| glycyrrhizinate oral | | |
| administration group II | | |

Note:
comparing the administration groups with the model group, p < 0.05 (*) and p < 0.01 (**). Comparing the combination oral administration groups with the lysozyme oral administration group, p < 0.05 (#) and p < 0.01 (##). Comparing the combination inhalation groups with the lysozyme inhalation group, p < 0.05(&) and p < 0.01(&&).

In the test, long-term inhalation of smoke caused a significant increase in the lung airway resistance and a significant decline in the lung function (reduced expiratory volume) in the animals. It could be seen from the test results that these administration groups reduced the airway resistance and increased the expiratory volume in the animals to varying degrees. By comparing the drug combination groups with the lysozyme-only groups, it was found that the glycyrrhizic acid-based substance enhanced the effect of the lysozyme, and in particular, when the dosage of the glycyrrhizic acid-based substance was relatively small, the synergistic effect was significant.

### Example 5: Protective Effects of Lysozyme and the Lysozyme Combinations Against Lipopolysaccharide-Induced Intestinal Barrier Dysfunction in Mice

Lipopolysaccharides could cause impaired intestinal function in animals. The protective effect of lysozyme and lysozyme combinations against lipopolysaccharide-induced intestinal barrier dysfunction in mice were studied in the present experiment.

Test drugs: lysozyme, monoammonium glycyrrhizinate, and dipotassium glycyrrhizinate.

Test animals: clean-grade male C57BL/6 mice, weighing 20 to 25 g.

Animal grouping and administration: The animals were randomly divided into a blank group, a model group, a lysozyme group (100 mg/kg), lysozyme+monoammonium glycyrrhizinate group A (100 mg/kg+2 mg/kg), lysozyme+monoammonium glycyrrhizinate group B (100 mg/kg+10 mg/kg), lysozyme+monoammonium glycyrrhizinate group C (100 mg/kg+20 mg/kg), and a lysozyme+dipotassium glycyrrhizinate group (100 mg/kg+2 mg/kg), with eight animals in each group. Each drug group was administered intragastrically by each dose of drugs every day, while the blank group and the model group were administered with normal saline. The administration was performed for 30 consecutive days.

Model establishment: After administration, the animals of each group except the blank group were intraperitoneally injected with 15 mg/kg of lipopolysaccharides, and the blank group was injected with normal saline. The animals were fasted for 12 hours after injection.

Determination of intestinal permeability: The animals of each group were administered intragastrically with 600 mg/kg of FITC-glucan (dissolved in a phosphate buffer), and after 4 hours, blood was taken from eyeballs. The content of the FITC-glucan in the serum was measured by means of fluorescence spectrometry (with an excitation wavelength of 480 nm and an emission wavelength of 520 nm), and the results were shown in Table 5.

Determination of expression of tight junction proteins in intestinal tissue: After the animals were sacrificed, jejunum tissue samples were taken for RNA extraction, the relative expression levels of occludin protein and Claudin-1 protein in the tissues were detected by quantitative PCR and the results were shown in Table 6.

TABLE 5
Effects of lysozyme and lysozyme combinations
on intestinal permeability

| | Content of FITC- |
|---|---|
| Group | glucan (μg/ml) |
| Blank group | 9.40 ± 1.45 |
| Model group | 35.06 ± 5.75 |
| Lysozyme group (100 mg/kg) | 27.53 ± 3.21** |
| Lysozyme + monoammonium glycyrrhizinate group A | 15.31 ± 2.08**## |
| (100 mg/kg + 2 mg/kg) | |
| Lysozyme + monoammonium glycyrrhizinate group B | 20.25 ± 2.44**## |
| (100 mg/kg + 10 mg/kg) | |
| Lysozyme + monoammonium glycyrrhizinate group C | 23.89 ± 2.52** |
| (100 mg/kg + 20 mg/kg) | |
| Lysozyme + dipotassium glycyrrhizinate group | 13.17 ± 1.14**## |
| (100 mg/kg + 2 mg/kg) | |

Note:
comparing the drug groups with the model group, p < 0.05 (*) and p < 0.01 (**). Comparing the drug combination groups with the lysozyme group, p < 0.05 (#) and p < 0.01 (##).

TABLE 6
Effects of lysozyme and lysozyme combinations on expression
of tight junction proteins in intestinal tissue

| | Relative | Relative |
|---|---|---|
| | expression | expression |
| | quantity of | quantity of |
| Group | Occludin mRNA | Claudin-1 mRNA |
| Blank group | 1.04 ± 0.12 | 1.07 ± 0.11 |
| Model group | 0.54 ± 0.09 | 0.73 ± 0.13 |
| Lysozyme group (100 mg/kg) | 0.69 ± 0.07** | 0.79 ± 0.16 |
| Lysozyme + monoammonium | 1.22 ± 0.24**## | 1.30 ± 0.19**## |
| glycyrrhizinate group A | | |
| (100 mg/kg + 2 mg/kg) | | |
| Lysozyme + monoammonium | 0.93 ± 0.16**## | 1.11 ± 0.15**## |
| glycyrrhizinate group B | | |
| (100 mg/kg +10 mg/kg) | | |
| Lysozyme + monoammonium | 0.67 ± 0.10* | 0.83 ± 0.17 |
| glycyrrhizinate group C | | |
| (100 mg/kg + 20 mg/kg) | | |
| Lysozyme + dipotassium | 1.07 ± 0.07**## | 1.00 ± 0.16**# |
| glycyrrhizinate group | | |
| (100 mg/kg + 2 mg/kg) | | |

Note:
comparing the drug groups with the model group, p < 0.05 (*) and p < 0.01 (**). Comparing the drug combination groups with the lysozyme group, p < 0.05 (#) and p < 0.01 (##).

The protective effects of the lysozyme and the lysozyme combinations against lipopolysaccharide-induced intestinal barrier dysfunction in mice were studied in the present experiment. After lipopolysaccharide injection, it was found that the content of the glucan in serum in the model group was significantly increased as compared with that in the blank group, indicating that a large amount of the glucan entered the blood through the intestine, that is, the intestinal permeability was increased; in addition, the expression of the two tight junction proteins in the intestine was decreased in the model group, further indicating that the intestinal barrier function was impaired. It was found through the present experiment that the lysozyme and the lysozyme combinations could significantly reduce the increase in the intestinal permeability induced by the lipopolysaccharide, significantly increase the expression of the tight junction proteins in the intestinal tissues, and had an obvious protective effect against intestinal barrier dysfunction. It was also found that the effect of the drug combination groups was obviously better than that of the lysozyme used alone.

In conjunction with a plurality of examples above, it is obvious that the lysozyme and the lysozyme combinations can inhibit cell damage caused by the novel coronavirus, inhibit the inflammatory factors expression induced by the novel coronavirus, ameliorate lung airway obstruction, increase the expiratory volume, ameliorate intestinal barrier dysfunction, and can reduce the novel coronavirus infection rate and the rate of progression to severe novel coronavirus pneumonia. The novel coronavirus pneumonia is still a new thing in the medical field. According to the current clinical knowledge, it has been shown that the patients have a high viral load in the early stage and obvious respiratory distress symptoms in the middle and late stages, and the intestinal barrier dysfunction leads to persistent and secondary infections, difficult rehabilitation, and increased infectivity in the patients, as well as a high mortality rate in severe patients, etc. Therefore, the lysozyme and the lysozyme combinations of the present invention are expected to prevent and treat the novel coronavirus pneumonia in various ways, and due to the good safety thereof, the dosage can be increased. The lysozyme and the lysozyme combinations are expected to become a better choice for preventing or treating the novel coronavirus pneumonia.

1.-52. (canceled)

53. A medicament for preventing or treating the novel coronavirus pneumonia COVID-19, or a food for assisting in preventing or treating the novel coronavirus pneumonia COVID-19, wherein the medicament or the food comprises lysozyme.

54. The medicament or the food according to claim 53, wherein the medicament or the food further comprises glycyrrhizic acid or a salt thereof.

55. The medicament or the food according to claim 54, wherein the weight ratio of the lysozyme to the glycyrrhizic acid or the salt thereof in the medicament or the food is 100:1 to 1:100, preferably 100:1 to 2:1, more preferably 100:1 to 5:1, and further preferably 100:1 to 10:1.

56. The medicament or the food according to claim 53, wherein the medicament further comprises a pharmaceutically applicable excipient.

57. The medicament or the food according to claim 53, wherein the food further comprises an additive suitable for food.

58. The medicament or the food according to claim 53, wherein the medicament further comprises an additional ingredient useful for preventing or treating the novel coronavirus pneumonia COVID-19.

59. The medicament or the food according to claim 58, wherein the additional ingredient useful for preventing or treating the novel coronavirus pneumonia COVID-19 in the medicament is one or more selected from an interferon, oseltamivir or a salt thereof, lopinavir, ritonavir, favipiravir, ribavirin, remdesivir, chloroquine or a salt thereof, hydroxychloroquine or a salt thereof, arbidol or a salt thereof, an interleukin inhibitor, a tumor necrosis factor inhibitor, a janus kinase inhibitor, a glucocorticoid, a protease inhibitor, or an intestinal microecological regulator.

60. The medicament or the food according to claim 53, wherein the dosage form of the medicament is an oral dosage form, an injection dosage form, or an inhalation dosage form.

61. The medicament or the food according to claim 53, wherein the release form of the medicament or the food is normal release, delayed release, sustained release, or controlled release.

62. A method for preventing or treating, or assisting in preventing or treating the novel coronavirus pneumonia COVID-19, comprising administering the medicament or the food according to claim 53 to a subject in need thereof, wherein the medicament or the food comprises an effective amount of lysozyme, or the medicament or the food comprises an effective amount of a combination comprising the lysozyme.

63. The method according to claim 62, wherein the combination further comprises glycyrrhizic acid or a salt thereof.

64. The method according to claim 63, wherein the lysozyme and the glycyrrhizic acid or the salt thereof in the combination exist separately in different pharmaceutical products or food products, or the lysozyme and the glycyrrhizic acid or the salt thereof coexist in the same pharmaceutical product or food product.

65. The method according to claim 63, wherein the weight ratio of the lysozyme to the glycyrrhizic acid or the salt thereof in the combination is 100:1 to 1:100, preferably 100:1 to 2:1, more preferably 100:1 to 5:1, and further preferably 100:1 to 10:1.

66. The method according to claim 62, with regard to the method for preventing or treating the novel coronavirus pneumonia COVID-19, the said combination comprising the lysozyme further comprises an additional ingredient useful for preventing or treating the novel coronavirus pneumonia COVID-19.

67. The method according to claim 66, wherein the additional ingredient useful for preventing or treating the novel coronavirus pneumonia COVID-19 is one or more selected from an interferon, oseltamivir or a salt thereof, lopinavir, ritonavir, favipiravir, ribavirin, remdesivir, chloroquine or a salt thereof, hydroxychloroquine or a salt thereof, arbidol or a salt thereof, an interleukin inhibitor, a tumor necrosis factor inhibitor, a janus kinase inhibitor, a glucocorticoid, a protease inhibitor, or an intestinal microecological regulator.

68. The method according to claim 62, wherein the subject is tested positive for COVID-19 novel coronavirus nucleic acid, or is clinically diagnosed with the novel coronavirus pneumonia COVID-19.

69. The method according to claim 62, wherein the subject further suffers from increased airway resistance, decreased expiratory volume, and/or impaired intestinal barrier function.

70. The method according to claim 62, wherein the administration route for the medicament or the food is oral administration, nasogastric feeding, injection, or inhalation.

71. The method according to claim 62, wherein the daily dosage of the lysozyme is 0.5 g to 20 g.

72. The method according to claim 62, wherein the medicament or the food is administered during the non-sleeping time at a dosage frequency of once every 1 to 24 hours, and preferably once every 1 hour, every 2 hours, every 4 hours, every 6 hours, every 8 hours, every 12 hours, or every 24 hours.
